(19) United States
(12) Patent Application Publication
(10) Pub. No.: US 2009/0306615 A1
(43) Pub. Date: Dec. 10, 2009

(54) TOP LAYER FOR AN ABSORBENT PRODUCT

(75) Inventor: Ken Olsson, Frolunda (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 12/306,147

(22) PCT Filed: Jun. 30, 2006
(86) PCT No.: PCT/SE06/00818

§ 371 Date: Dec. 22, 2008

(51) Int. Cl. A61F 13/15

(52) U.S. Cl. 604/367; 604/378

(57) ABSTRACT

An absorbent product such as a panty liner, incontinence pad, sanitary towel or the like comprising a top layer consisting of a first material layer and a second material layer, the first material layer being attached directly to the second material layer. The first material layer has a first part and a second part, where the first part and/or the second part contains openings, and where the first part is folded over the second part along a fold line, the first material layer being attached directly to the second material layer adjacent to at least one line extending in the longitudinal direction in an inner joining zone. A method for the manufacture of an absorbent product of this kind.

### TECHNICAL FIELD

The invention relates to an absorbent product such as a panty liner, incontinence pad, sanitary towel or the like comprising a top layer consisting of a first material layer and a second material layer. The first material layer is attached directly to the second material layer. The absorbent product exhibits two lines extending in the longitudinal direction, which divide the product in a lateral direction in such a way that the product exhibits a first side zone, a second side zone and positioned between them a central zone. The invention also relates to a method for the manufacture of an absorbent product of this kind.

### BACKGROUND ART

In previously disclosed absorbent products, the top layer comprises a material layer that is liquid-permeable in order to be able to transport liquid from the top layer to the subjacent absorption body. The liquid can be menstrual fluid or urine, and the choice of the material used for the top layer depends on which of these liquids the absorbent product is primarily intended to take up. When choosing the material, consideration must also be given to the required suitability of the top layer having regard for the need for the wearer to experience the material as comfortable against his/her body, and for the wearer to experience a sensation of dryness after the liquid has been excreted. A hydrophobic material is already known to give such a dry sensation. Also previously disclosed is the embodiment of a water-impermeable material layer with holes in order to make the top layer permeable to air and water, at the same time as the water-impermeable material in other respects prevents the reflux of moisture from the absorption body.

The problems associated with the choice of material for the top layer are particularly great in the side zones, because the side zones of the absorbent product make contact at least partially with a wearer's groin. The nature of the problem is that the groin moves relative to the side zones, which can give rise to chafing, and that the side zones become crumpled. Movement by the wearer also causes the absorbent product to describe simultaneous movement in the side zones, which imposes high demands on the material in order for the material not to rupture. The movement in the side zones is usually less than the movement in the central zone, which can give rise to chafing on the wearer if an inappropriate material is used in the side zones.

WO 03/015681 depicts an absorbent product comprising a top layer of a film or nonwoven material containing a number of holes, the holes being larger in the central zone than in the side zones. The small holes in the side zones are provided to give the material a softer feel compared with a non-perforated material. One problem with holes is that the holes are always open and permit excessive reflux of moisture from the absorption body. Another problem is that the holes permit leakage of material lying loosely under the top layer, for example superabsorbents (SAP), fibres, odor inhibitors, etc. Superabsorbents are small balls or grains of an absorbent material lying loosely in the absorption body, which can be transformed into a gel-like substance by the absorption of water. Any material lying loosely that has found its way out through the holes ends up between the absorbent product and the wearer's skin and can cause discomfort for the wearer, for example in the form of chafing. The above-mentioned problems are especially great in the side zones of the absorbent product, because the movement of the side zones affects the absorption body in such a way that SAP and liquid tend to be transported out through the holes by the movement in the side zone. Making the holes so small that the superabsorbents are not able to leak out through the holes, or so small that the moisture reflux problem is reduced, would reduce the transport of liquid from the top layer to the absorption body, that is to say the admission capacity of the top layer to the subjacent material, to an unacceptably low level. Similar problems exist for all particles that risk leaking out through the holes, for example the above-mentioned particles.

A wish and a need accordingly remain for an absorbent product, in which the above problems are overcome by means of a top layer having improved characteristics in the side zones as far as the minimal leakage of loosely lying material is concerned. A further wish is for the top layer, at least in the side zones, to present a soft and comfortable surface to the wearer. Additional desirable advantages are for the side zones to possess a high degree of air permeability in order to reduce the amount of moisture present in the groin of a wearer. There is also a desire for a simple and inexpensive method for the manufacture of such an absorbent product.

### DISCLOSURE OF INVENTION

The invention proposes to solve the above-mentioned problems by means of an absorbent product such as a panty liner, incontinence pad, sanitary towel or the like comprising a top layer consisting of a first material layer and a second material layer. The first material layer is attached directly to the second material layer. The absorbent product exhibits two lines extending in the longitudinal direction, which divide the product in a lateral direction in such a way that the product exhibits a first side zone, a second side zone and positioned between them a central zone.

The invention is characterized in that the first material layer comprises a first part and a second part, where the first part and/or the second part contains openings, and where the first part is folded over the second part along a fold line, the first material layer being attached directly to the second material layer adjacent to at least one of the lines extending in the longitudinal direction in an inner joining zone.

The expression “openings” is used here to denote holes or slits, and embodiments of the invention that are described below in conjunction with the use of holes are also eligible for slits.

The expression “fold line” is used here to denote an envisaged line, along which the first material layer is folded. The fold line thus need not take the form of a variation present in the material layer, for example a compressed part or the like, although it is not precluded from including such a variation.

The expression “absorbent product” is used here essentially to denote a material or a material composition with the ability to retain liquid for a predetermined period.

The subdivision of the absorbent product into zones and sections is of a theoretical nature without distinct boundaries and is performed for the sole purpose of facilitating understanding of the invention. The longitudinally extending lines can thus be straight or curved, depending on the design of the product, in conjunction with which the central zone is not restricted to a square or rectangular geometry, but can exhibit any appropriate geometry, for example circular, oval, triangular, etc. The side zones thus have a corresponding design at the interface with the central zone and in a corresponding manner follow the contour of the absorbent product, that is to say they follow the edge part of the absorbent product.

The expression “adjacent to” is used here to denote that the inner joining zone either includes or excludes the longitudinally extending line. In a preferred embodiment, the distance between the fold line and the inner folding zone consists of a maximum of two-thirds of the distance from the fold line to an edge line present in the absorbent product. The greater the distance from the fold line, the more the folded material layer will overlap the second material layer in the central zone. An appropriate distance from the fold line thus depends on the size of the product and the reason why an overlap is desirable. The folded overlap forms a space between the folded parts of the first material layer. The space can be filled with elastic, for example, in order to form the product, or an absorption material, or a skin care substance, or an odor-inhibiting substance, or some other optional material. Examples of elastic elements are elastic threads, elastic pieces of foam, elastic layers or laminates, etc. The size of the overlap, that is to say the distance between the fold line and the inner joining zone, thus depends on the envisaged area of application and can thus be greater than two-thirds, should the need arise.

The joining zone can be joined continuously or discontinuously and can be joined by means of an optional joining method. Joining can take place, for example, by gluing, or by welding in the form of ultrasonic or laser, or by mechanical joining, for example in the form of embossing or compression, etc., or by some other appropriate method of joining.

One advantage of the invention is that loose particles present under the top layer are prevented from finding their way out through the top layer via the holes, at the same time as holes are permitted in the first material layer, which confers the advantages described below.

Another advantage of the invention is that the first material layer can be manufactured separately and the second material layer can be manufactured separately, after which they are joined together in the desired manner.

According to one embodiment of the invention, the first material layer comprises two subsidiary layers arranged to either side of the second material layer, each subsidiary layer comprising a first part and a second part, where the first part and/or the second part contains holes, and where the first part is folded over the second part along a fold line, the first material layer being attached directly to the second material layer adjacent to the lines extending in the longitudinal direction in inner joining zones. The expression “to either side” is used here to denote a positioning of the subsidiary layers essentially in the same plane as the second material layer.

According to one embodiment of the invention, the first part contains holes and the second part lacks holes, which first part is folded over the second part in such a way that the holes are covered by the second part. The first part is arranged on the absorbent product in such a way that it faces towards a wearer during use. One advantage of this embodiment is that the top layer, as a consequence of the holes, takes on a textile feel in accordance with the prior art, but that loose particles present under the top layer are prevented from finding their way out through the top layer via the holes.

According to another embodiment of the invention, the second part contains holes and the first part lacks holes, which first part is folded over the second part in such a way that the holes are covered by the first part. The first part is arranged on the absorbent product in such a way that it faces towards a wearer during use. One advantage of this embodiment is that the top layer, as a consequence of the holes, permits the transport of a high volume of air between a subjacent material and the space that is formed between the first part and the second part, but that loose particles present under the top layer are prevented from finding their way out through the top layer via the holes. The space has the advantage that the space buffers the air, which is then able to diffuse out through an air-permeable first part.

Further advantages of this embodiment are that loose particles, for example superabsorbents or particulate odor inhibitors or the like, are able to pass through the holes into the space, where they can perform their absorbent effect in the envisaged manner, instead of leaking into the surroundings.

The superabsorbents swell on contact with liquid, which means that superabsorbents which absorb liquid in the space form a thickened side edge, which has a positive effect on the absorbent product in that a barrier is built up between the first material layer and the second material layer. The barrier prevents liquid from spreading out towards the edges from the second material layer.

Odor inhibitors absorb or adsorb malodorous substances in dry and moist air. The odor inhibitors that are present in the space are thus able to continue to act on the air that is being buffered.

According to one embodiment of the invention, both the first part and the second part contain holes. When the first material layer is folded over itself, the holes in the first part will essentially only partially overlap the holes in the second part. Because the holes overlap one another only partially, the channels that are formed by two partially overlapping holes are sufficiently small to prevent loose particles present under the top layer from escaping, at the same time as the holes in the first part permit the optimal transport of liquid and air to the second part, where the holes in the second part provide optimal transport of liquid and air through the second part. In the same way, the holes in the second part permit the transport of liquid and air to the first part, which, via the holes, gives optimal liquid and air transport through the first part. The first part or the second part is arranged on the absorbent product in such a way that it faces towards a wearer during use. One advantage of this embodiment is that the top layer, as a consequence of the holes, takes on a textile feel in accordance with the prior art, but that loose particles present under the top layer are prevented from finding their way out through the top layer via the holes.

According to one embodiment of the invention, the extent of the first part in the lateral direction is smaller than the second part, and the opposite situation prevails according to another embodiment, such that the extent of the second part in the lateral direction is smaller than the first part. An interjacent joining zone is present here adjacent to the edge of the shorter part, that is to say at some point between the fold line and the opposing outer edge of the longer part, which joins together the first part and the second part. The expression “short” is used here to denote an extent in the lateral direction. In this embodiment, the first material layer contains holes only under the folded-over common area between the fold line and the interjacent joining zone. The part of the first material layer that is present between the interjacent joining zone and the outer edge of the first material layer lacks holes, as it might otherwise be possible for material to leak out. One advantage of this embodiment is that the consumption of material is smaller, at the same time as the above-mentioned advantages are achieved. A further advantage is that the space that is formed between the fold line and the interjacent joining zone can be utilized to provide the first material layer with an elastic arrangement in order to lift up a part of the first material layer in the form of a barrier. Another alternative is to fill the space totally or partially with a specially adapted material, for example an absorbent material, in order to absorb liquid or a volume-increasing material to build up a barrier in accordance with the above.

According to one embodiment, the absorbent product includes a backing layer, to which the first material layer is attached via an outer joining zone. The first material layer includes an outer edge part on the opposite side of the fold line, and the first material layer is advantageously attached to the backing layer adjacent to this outer edge part. The absorbent product can also include an absorption body between the top layer and the backing layer. The outer joining zone in this case can also include an outer part of the absorption body.

According to one embodiment of the invention, the absorbent product comprises a receiving layer, to which the first material layer and the second material layer are attached via the inner joining zone. One advantage of this embodiment is that the receiving layer and the top layer can be manufactured as a unit, which can subsequently be applied to any desired core element, for example consisting of an absorption body and/or a backing layer, etc.

The invention also relates to a method for the manufacture of an absorbent product, such as a panty liner or a sanitary towel, comprising a top layer containing a first material layer and a second material layer, which first material layer is attached directly to the second material layer. The method is characterized in that the first material layer consists of a first part and a second part, where the first part or the second part contains holes, and where the first part is folded over the second part along a fold line in such a way that the part provided with holes is covered by the part which lacks holes, the first material layer being attached directly to the second material layer adjacent to the fold line via an inner joining zone.

One advantage of the method is that the first material layer can be manufactured separately in the form of a web, in which holes are made only in a part of the first layer, which offers the possibility of being able to control the position of the perforated part by simple means in relation to the non-perforated part. If perforation were to be performed in situ after the top layer had been arranged on the core element of the absorbent product, it would be very difficult to make holes only in one of the folded-over parts, and particularly difficult, of course, when there is a desire for holes on that part of the top layer that faces away from the wearer during use.

In conjunction with the manufacture of a separate top layer according to the invention, it is easy to use an optionally prefabricated first layer on the basis of desired parameters in respect of the number of holes, the width of the first part and the second part, etc., and then to fold the first material along the line which separates the first part from the second part. The method thus provides the manufacturer with the opportunity to resort to the use of a subcontractor with simple specifications for a flat layer to the desired design. It can be stated by way of example that this freedom of choice means that the manufacturer of the absorbent product can select an appropriate width for the first part and the second part respectively in order to permit one fold, two folds, three folds or more folds. A plurality of folds gives a thickness in the first material layer with the ability to form a barrier with the associated advantages indicated above.

The absorption body is appropriately manufactured from a suitable fibre material, in the form of natural or synthetic fibres having absorbent properties, or a mixture of natural fibres and synthetic fibres or other absorbent material of a previously disclosed kind that are suitable for use in sanitary towels, incontinence pads and panty liners, for example. The absorption body can also contain a predetermined proportion, for example 20-60%, of superabsorbent materials, for example polymer materials in the form of particles, fibres, flakes or similar, which have the ability to absorb and to chemically bind liquid equivalent to several times their own weight while forming an aqueous gel. This provides a very high water-absorbent capacity in the finished product.

It must also be noted that the absorption body can exhibit different forms, for example an essentially elongated and rectangular form, or alternatively some other more irregular form, for example hourglass or triangular form. The absorption body also preferably has rounded edges.

The liquid-permeable first and/or the second material layer (hereinafter referred to as the top layer) preferably consists of one or more layers of one or other of the following materials: a fibrous material, for example a soft nonwoven material, plastic film, mesh, open-celled foam, material laminate, etc. The top layer is preferably fully or partially perforated, that is to say slits or holes are made in the top layer. The top layer can appropriately consist of a perforated plastic film, for example a thermoplastic plastic material such as polyethylene or polypropylene, or a mesh-like layer of synthetic or textile material. Synthetic mono-, bi-, or multi-component fibres, made of polymers such as polyethylene, polypropylene, polyester, nylon or the like, are preferably used as a nonwoven material. Mixtures of different types of fibres can also be used for the aforementioned nonwoven material. The invention is not, however, restricted in principle to use only for top layers which consist of nonwoven material, but can also be applied in conjunction with the processing of other materials, for example films made of thermoplastics such as polyethylene or polypropylene.

The invention can also be implemented with a top layer which consists of different types of laminates or combinations of laminates and/or single layers. For example, the top layer can consist of a number of different laminates or single layers which cover parts of the surface of the product. In the event that the product consists of a plurality of laminates or single layers, for example divided up into a plurality of longitudinal parts having different sections, these different sections can consist of different materials and can possess different characteristics. For example, each section can then have different types of perforation, hole positioning, dimensions, hydrophobicity, etc. The different sections can be joined together by means of ultrasonic welding in a previously disclosed manner that is not described here in detail.

The liquid-permeable top layer is preferably manufactured from a material that exhibits characteristics such as dryness and softness during the time when the absorbent product is being worn, because this top layer is in contact with the wearer's body. It is also desirable for the top layer to have a soft and textile-like surface which remains dry, even in the event of repeated wefting. The top layer can consist of a nonwoven material, for example, with a soft and smooth surface, such as a spunbond material made from polypropylene fibres. A perforated, hydrophobic nonwoven material may be used in order to permit the surface that is closest to the wearer's body to be kept dry, in conjunction with which holes are formed in the material that are larger than the distance between the fibres in the material. In this way, liquid can be led down through the holes in the top layer to the subjacent absorption body. Other examples of materials for the top layer are perforated plastic films such as a perforated polyester film. The top layer can be joined together with the subjacent backing layer and the absorption body, for example by means of adhesive, ultrasonic jointing or by means of some form of thermal bonding.

The top layer can contain small perforations which surround the holes in the top layer with the intention of further increasing the intake of liquid and the acquisition characteristics.

It is desirable for the thickness of the top layer to be as great as possible, because a great thickness has a positive effect on the acquisition.

The top layer can also be a three-dimensional laminate of nonwoven and plastic film or a carded, thermally bonded material based 100% on polypropylene. The plastic film can be hydrophilic, pre-perforated (with small holes) and manufactured from a mixture of polyethylene and polypropylene. The nonwoven materials can have a weight per unit area in the range from 12-100 gsm, and in particular in the range from 15-60 gsm.

The nonwoven part of the top layer can also be a spunbond nonwoven material, an air-thru nonwoven material, a spunlace nonwoven (hydroentangled) material, a meltblown nonwoven material, or a combination of these. The raw material can be polypropylene (PP), polyethylene (PE) polyester (PET), polyamide (PA), or a combination of these. If a combination is used, this can be a mixture of fibres from different polymers, although each fibre can also contain different polymers (e.g. PP/PE bi-component fibres or PP/PE copolymers). Where appropriate, the plastic film can consist of PE or PP, PET, PLA or amyl (or any other thermoplastic polymer), or a mixture or copolymers of the aforementioned polymers.

The perforated top layer can also be manufactured from a single layer of material, such as a nonwoven material or a film (as described above).

The holes in the top layer can be oval and slightly elongated in the direction of the machine. The holes can be round/circular or oval in the direction of the machine or the transverse direction. The holes can also be replaced by slits, which by definition differ from the holes in that the slits do not constitute constant openings, but instead are through going incisions in the material layer. The slits are opened and closed by movement in the material and offer the advantage that material lying loosely under the top layer does not leak out to the same extent as in the case of holes, but that the top layer maintains a good ability to breathe and that the slits give a textile feel. The slits are preferably from 2 mm up to 15 mm in length, and preferably lie in the range from 3-10 mm. The length of the slits is measured along the boundary surfaces of the slits in a direction essentially perpendicular to the thickness of the top layer and when the slit is in its closed state. The length of the slits and the distance between the slits and the direction of the slits varies depending on the material in the top layer, because the natural tendency of the slits to open depends on the characteristics of the material present in the top layer.

The slits are arranged in the top layer with a mutual distance between the slits having a size in the order of 5-15 mm, although this is dependent on a range of factors, for which reason the distance between the slits can vary depending, among other things, on the material in the top layer and the length of the slits and the direction of the slits. This distance must be sufficiently great between the slits to prevent the top layer from being torn apart when the wearer moves, and sufficiently great to allow the slits to close in the desired manner under the influence of other slits, although at the same time sufficiently small for the ability to breathe and the liquid permeability to remain at an acceptable level. The durability of the top layer is largely governed, however, by the relationship between the surface containing slits and the surface without slits for a given material strength, where the distance between the slits is a subset of the parameters for the durability.

The backing layer is preferably liquid-impermeable (or at least possesses high resistance to penetration by liquid) and is thus so arranged as to prevent any leakage of excreted fluid from the product. The backing layer, on the other hand, may be executed so that it is vapour-permeable. For this purpose, the backing layer may be manufactured from a liquid-impermeable material which consists appropriately of a thin and liquid-proof plastic film. For example, plastic films of polyethylene, polypropylene or polyester can be used for this purpose. Alternatively, a laminate of nonwoven and plastic film or other suitable material layers can be used as a liquid-proof backing layer. In a previously disclosed manner, the under side of the backing layer can be provided with beads of adhesive or some other previously disclosed attachment means, which can then be utilized for the application of the product to an item of clothing. The product can also be provided with wings, that is to say folding flaps which, in a previously disclosed manner, are arranged along the sides of the product and can be utilized in conjunction with the application of the product.

The product also includes a further layer of material in the form of a receiving layer (also referred to as an acquisition layer, an admission layer and a distribution layer, depending on the function of the material). The receiving layer can be in the form of a wadding material having an appropriately specified thickness and resilience, which is intended to be positioned between the absorption body and the top layer. The admission layer possesses essentially the same dimensions as the top layer, with the exception of its thickness, however, which can deviate from the thickness of the top layer. It is also possible to establish that the admission layer can consist of materials other than wadding material. For example, it may consist of a so-called airlaid material, which is usually based on cellulose fibres. The admission layer can also incorporate fibrous materials in order to impart an appropriately balanced rigidity to it. The admission layer can also incorporate an appropriate quantity of thermoplastic fibres in order to permit ultrasonic welding.

The admission layer can appropriately be a porous, elastic, relatively thick layer of material, for example in the form of a fibrous wadding material, a carded fibre wadding, a tow material, or some other kind of bulky and/or resilient fibre material with a high instantaneous liquid intake capacity that is capable of storing liquid temporarily before it is absorbed by the subjacent absorption body. The admission material can also be in the form of a porous foam material. It can also consist of two or more layers of material. According to a preferred embodiment, the admission layer can extend towards the lateral edges of the product, that is to say it possesses essentially the same form as the top layer. In this way, advantages can be achieved in respect of liquid distribution, edge sealing, etc.

When manufacturing the absorbent product, the top layer is joined to the backing layer and can also be joined to the receiving layer and/or the absorption body. Joining can take place by gluing, or by welding by means of ultrasonic or laser, or by mechanical joining, for example in the form of embossing or compression, etc., or by some other appropriate method of joining, for example thermal bonding.

It must be stated, however, that the choice of material and the thickness of the material layer may change in the future in the event of changed manufacturing methods and new material combinations, as a consequence of which the invention is not restricted to the materials and material combinations indicated above.

### BRIEF DESCRIPTION OF DRAWINGS

The invention is described below in conjunction with preferred illustrative embodiments and the accompanying drawings, in which

FIG. 1 depicts schematically a view from above of an absorbent product in accordance with the invention;

FIG. 2 depicts schematically a sectional view along the line A-A in FIG. 1 according to a first embodiment of the invention;

FIG. 3 depicts schematically a sectional view along the line A-A in FIG. 1 according to a second embodiment of the invention;

FIG. 4 depicts schematically a sectional view along the line A-A in FIG. 1 according to a third embodiment of the invention;

FIG. 5 depicts schematically a sectional view along the line A-A in FIG. 1 according to a fourth embodiment of the invention;

FIG. 6 depicts schematically a sectional view along the line A-A in FIG. 1 according to a fifth embodiment of the invention; and where

FIG. 7 depicts schematically a sectional view along the line A-A in FIG. 1 according to a sixth embodiment of the invention.

### MODE(S) FOR CARRYING OUT THE INVENTION

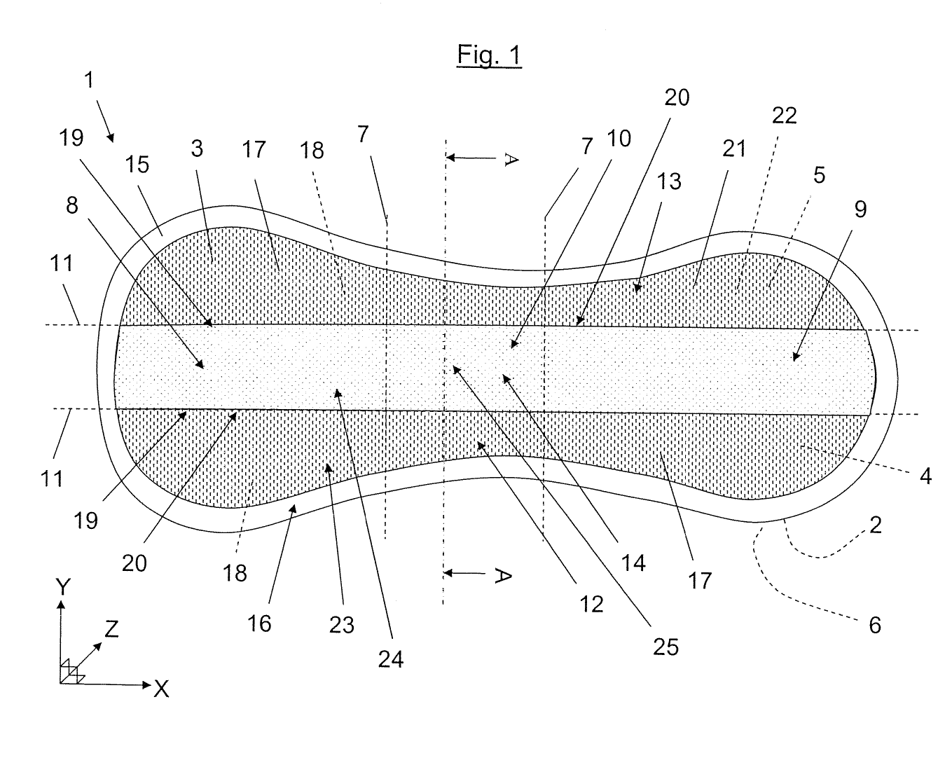

FIG. 1 depicts schematically a view of the top layer of an absorbent product 1 according to the invention. The absorbent product 1 here consists of a sanitary towel, which can be used as undergarment protection in conjunction with menstruation or light incontinence. The absorbent product 1 is positioned in a plane position and exhibits in this plane an extent in the longitudinal direction, the X-axis in the Figure, and in the longitudinal direction, the Y-axis in the Figure, and has a thickness perpendicular to the plane, the Z-axis in the Figure. When wearing the absorbent product 1, the absorbent product 1 is formed around the lower abdomen of a wearer.

The absorbent product 1 comprises a backing layer 2, a top layer 3 and between them an absorption body 4. The absorption body 4 has a first surface 5 and a second surface 6, in conjunction with which the backing layer 2 is arranged over the second surface 6 of the absorption body 4, and the top layer 3 is arranged over the first surface 5 of the absorption body 4. Depicted in FIG. 1 are two laterally extending lines 7, which theoretically divide the product in the longitudinal direction into a rear section 8, a front direction 9 and between them a central section 10. Depicted in FIG. 1 are two lines 11 extending in the longitudinal direction, which divide the product in the lateral direction described by a first side zone 12 and a second side zone 13 and positioned between them a central zone 14. FIG. 1 shows that the absorbent product 1 is bounded by a peripheral edge part 15. FIG. 1 also shows that the top layer 3 is joined to the backing layer 2 in outer joining zones 16 which coincide with the peripheral edge part 15.

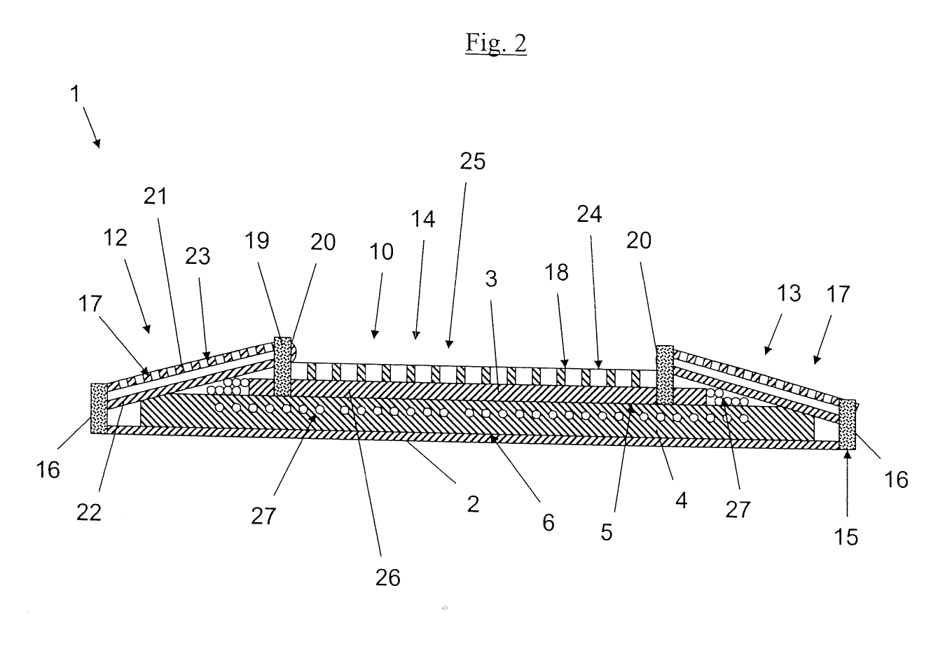

The top layer 3 comprises a first material layer 17 in the side zones 12, 13 and a second material layer 18 in the central zone 14. The first material layer 17 is divided into two subsidiary layers arranged to either side of the second material layer 18, which subsidiary layers respectively cover the side zones 12, 13. The first material layer 17 and the second material layer 18 are thus positioned essentially next to one another in the lateral direction and are joined in inner joining zones 19 along the lines 11 extending in the longitudinal direction. The first material layer 17 is folded over itself along a fold line 20 at least once, that it to say it is at least double-folded, and it comprises a first part 21 and a second part 22. According to the invention, either the first part 21 or the second part 22 contains a number of holes 23. The part 21, 22 of the first material layer 17 which lacks holes 23 is intended to prevent loose particles that are present under the top layer 3 from migrating out from the absorbent product 1 via the holes 23. Different embodiments of the first material layer 17 are described below in conjunction with FIGS. 2-7.

The second material layer 18 advantageously contains holes 24 and possesses characteristics that are particularly adapted to the so-called wet area 25. The wet area 25 is an area in the absorbent product 1 which substantially coincides with the part of the absorbent product 1 that is formed by the section through the central zone 14 and the central section 9 and can vary depending on the primary area of application of the absorbent product 1, for example light incontinence protection or menstruation protection. When using the absorbent product 1 as an incontinence pad, the wet area 25 consists of the part of the absorbent product 1 which, when it is being used by a wearer, lies facing towards and in close proximity to the wearer's urethra. The wet area 25 thus constitutes the part of the absorbent product 1 that will receive the largest flow of liquid in conjunction with the excretion of liquid waste by the wearer. In the case of menstruation protection, the centre of the wet area 25 can be displaced somewhat in relation to the urethra in such a way that the wet area 25 is positioned against that part of a women's lower abdomen from which the flow of menstruation fluid is discharged.

FIG. 2 depicts schematically a sectional view along the line A-A in FIG. 1 according to a first embodiment of the invention. FIG. 2 shows that the side zones 12, 13 comprise the first material layer 17 double-folded and including the first part 21 containing a number of holes 23 and the second part 22 which lacks holes. The first part 21 is positioned on the top side of the absorbent product 1, that is to say on that part of the absorbent product 1 which is intended to face towards a wearer during use. The perforated first part 21 is intended here to give the material in the side zones 12, 13 a textile and comfortable feel for the wearer. The second part 22 is arranged between the first part 21 and the subjacent laminated structure consisting of a receiving layer 26, the absorption body 4 and the backing layer 2.

FIG. 2 shows that the absorption body 4 contains superabsorbents 27 (hereinafter referred to as SAP) in the form of small granules. FIG. 2 also shows that the SAP granules 27 have migrated from the absorption body 4 in such a way that they are lying freely under the top layer 3 in the side zones 12, 13. The SAP granules migrate in the absorption body depending on the construction and structure of the absorption body. The other part 22 of the first material layer 17 prevents the SAP granules 27 from escaping through the top layer 3 into the side zones 12, 13, which means that the top layer 3 can contain holes 23 for imparting the textile feel.

As mentioned above, the absorbent product 1 includes a receiving layer 26 positioned between the top layer 3 and the absorption body 4. The inner joining zones 19 extend in the Z-axis through both the first material layer 17 and the second material layer 18 and at least partially through the receiving layer 26. An advantage is achieved in the manufacturing process by attaching the top layer 3 to the receiving layer 26, or to the absorption body 4 when no receiving layer 26 is present, because the receiving layer 26 at the time of manufacture is advanced in a well-defined path, which imparts a fixed position to the top layer 3 relative to the receiving layer 26 in a finished element. A further advantage is that the produced element can be attached simply to the so-called core element, which consists of the other layers present in the absorbent product 1, that is to say the backing layer 2 and the absorption body 4 or, in an appropriate case, the backing layer 2 alone. The receiving layer 26 consists of a material with an open structure which permits the rapid transport of liquid from the top layer 3 to the subjacent absorption body 4. The receiving layer 26 can possess a high or a low distribution capacity, although it will always transport liquid effectively from the top layer 3 to the absorption layer 4. The task of the receiving layer 26 is to ensure that the wearer experiences a feeling of dryness once liquid has been transported away from the top layer 3.

FIG. 2 shows that the first part 21 and the second part 22 have identical lengths, and that the inner joining zones 19 bond the top layer 3 and the receiving layer 26 together at the point where the first material layer 17 has its fold line, that is to say along the longitudinally extending lines 11. The outer joining zone 16 bonds the first part 21, the second part 22 and the backing layer 2 together. The outer joining zone 16 can also contain the absorption body 4, in which case the edge part 15 possesses a softer feel than when only the top layer 3 and the backing layer 2 form the edge part 15.

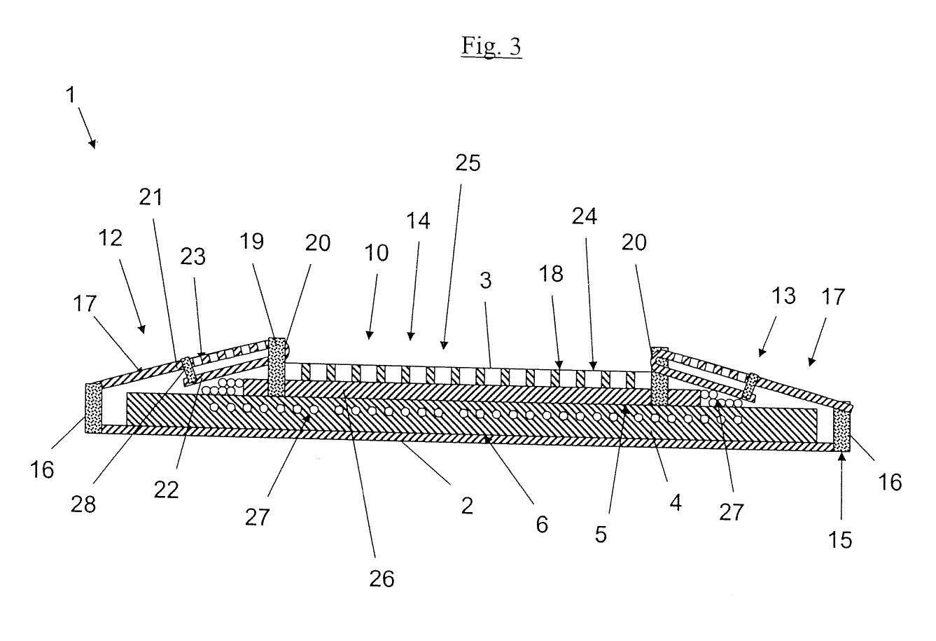

FIG. 3 depicts schematically a sectional view along the line A-A in FIG. 1 according to a second embodiment of the invention. FIG. 2 and FIG. 3 are identical apart from the execution of the first material layer 17. FIG. 3 shows that the first part 21 is longer than the second part 22. The inner joining zones 19 bond the top layer 3 and the receiving layer 26 together at the point where the first material layer 17 has its fold line 20, that is to say along the longitudinally extending lines 11. The outer joining zone 16 bonds the first part 21 and the backing layer 2 together. The outer joining zone 16 can also contain the absorption body 4, in which case the edge part 15 possesses a softer feel than when only the top layer 3 and the backing layer 2 form the edge part.

FIG. 3 shows that the first part 21 and the second part 22 are joined together via interjacent joining zones 28 adjacent to the area where the second part 22 ends. The second part 22 ends at a predetermined distance between the fold line 20 and the outer joining zone 16. The first part contains the holes 23 only for the same extent as the second part 22, that is to say between the fold line 20 and the interjacent joining zone 28. One advantage of this embodiment is that at least parts of the first material layer 17 impart a textile feel, but that the consumption of material for the top layer 3 is reduced.

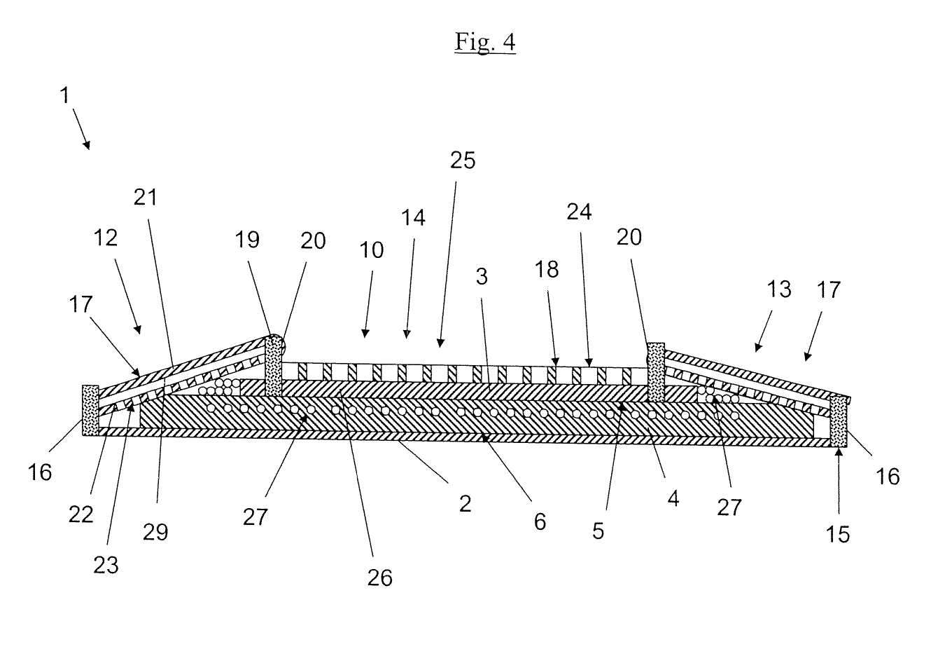

FIG. 4 depicts schematically a sectional view along the line A-A in FIG. 1 according to a third embodiment of the invention. FIG. 2 and FIG. 4 are identical apart from the execution of the first material layers. FIG. 4 shows that the first part 21 and the second part 22 have identical lengths, and that the inner joining zones 19 bond the top layer 3 and the receiving layer 26 together at the point where the first material layer 17 has its fold line 20, that is to say along the longitudinally extending lines 11. The outer joining zone 16 bonds the first part 21, the second part 22 and the backing layer 3 together. The outer joining zone 16 can also contain the absorption body 4, in which case the edge part 15 possesses a softer feel than when only the top layer 3 and the backing layer 2 form the edge part.

FIG. 4 shows that the first part 21 lacks holes, and that the second part contains the holes 23. The first part 21 is positioned on the upper side of the absorbent product, that is to say on that part of the absorbent product 1 which is intended to face towards a wearer during use. The second part 22 is arranged between the first part 21 and the subjacent laminated structure consisting of the receiving layer 26, the absorption body 4 and the backing layer 2. The perforated second part 22 is intended here to give the material in the side zones 12, 13 increased air permeability, at the same time as the first part 21 prevents loose material, for example SAP granules, from leaking out through the top layer 3. A further advantage of this embodiment is that the SAP granules that have migrated out through the holes can continue to be active in the space 29 that is formed between the first part 21 and the second part 22.

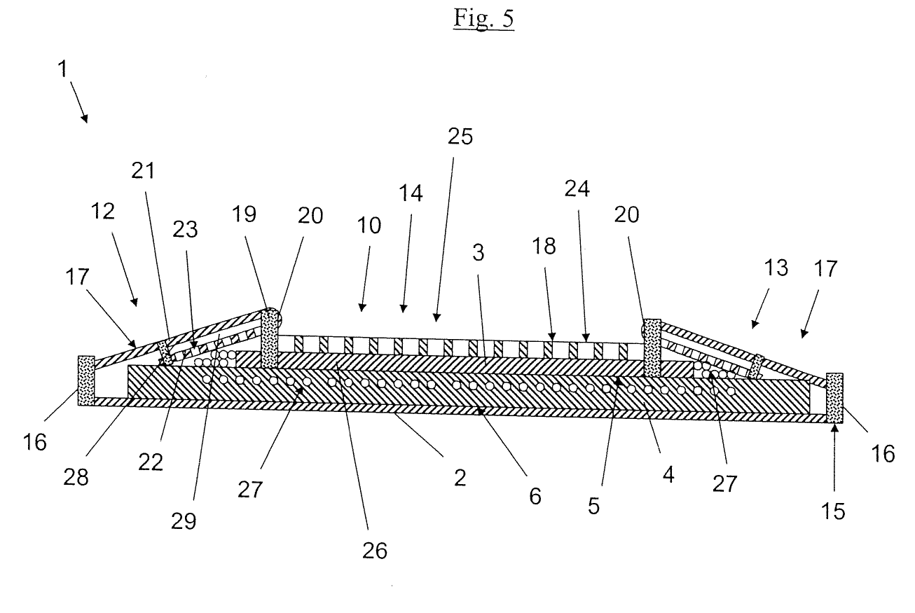

FIG. 5 depicts schematically a sectional view along the line A-A in FIG. 1 according to a fourth embodiment of the invention. FIG. 5 and FIG. 4 are identical apart from the execution of the first material layer 17. FIG. 5 shows that the first part 21 is longer than the second part 22. The inner joining zones 19 bond the top layer 3 and the receiving layer 26 together at the point where the first material layer 17 has its fold line 20, that is to say along the longitudinally extending lines 11. The outer joining zone 16 bonds the first part 21 and the backing layer 2 together. The outer joining zone 16 can also contain the absorption body 4, in which case the edge part 15 possesses a softer feel than when only the top layer 3 and the backing layer 2 form the edge part.

FIG. 5 shows that the first part 21 and the second part 22 are joined together via interjacent joining zones 28 adjacent to the area where the second part 22 ends. The second part 22 ends at a predetermined point between the fold line 20 and the outer joining zone 16. The second part 22 thus contains the holes 23 between the fold line 20 and the interjacent joining zone 28. Apart from the advantages referred to in conjunction with FIG. 4, a further advantage achieved with this embodiment is that the consumption of material for the top layer is reduced.

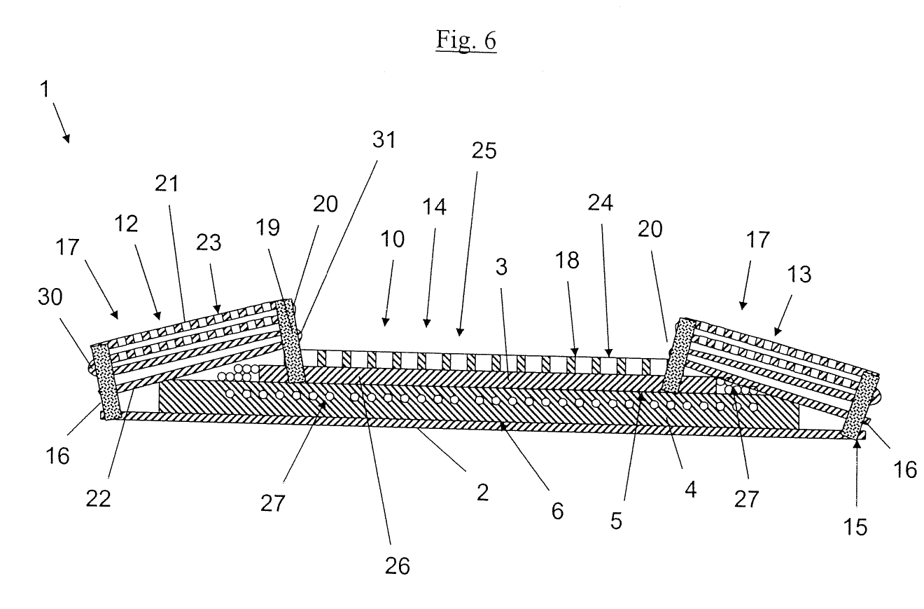

FIG. 6 depicts schematically a sectional view along the line A-A in FIG. 1 according to a fifth embodiment of the invention. FIG. 6 is identical with FIGS. 2 and 4 as far as concerns the length of the first and the second material layer respectively. FIG. 6 shows that the first part 21 is folded double and that the second part 22 is folded double. The inner joining zones 19 bond the top layer 3 and the receiving layer 28 together at the point where the first material layer has its fold lines 20, that is to say along the longitudinally extending lines 11.

The outer joining zone 16 bonds together the following: the overlapping parts of the first part 21 adjacent to a fold line 30 which extends essentially along the edge line 15 and the overlapping parts of the second part 22 adjacent to a fold line 31 which extends essentially along the edge line 15; and the backing layer 2. The outer joining zone 19 can also contain the absorption body 4, in which case the edge part 15 possesses a softer feel than when only the top layer and the backing layer form the edge part.

FIG. 6 shows that the first part 21 contains the holes 23, and that the second part 22 lacks holes. The first part 21 is positioned on the upper side of the absorbent product 1, that is to say on that part of the absorbent product 1 which is intended to face towards a wearer during use. The second part 22 is arranged between the first part 21 and the subjacent laminated structure consisting of the receiving layer 26, the absorption body 4 and the backing layer 2. The perforated first part 21 is intended here to give the material in the side zones 12, 13 an increased textile feel. The double-folded first and second parts 21, 22 impart increased thickness to the side zones 12, 13, which has the advantage that the first material layer 17 forms barriers to prevent lateral leakage, that is to say the leakage of liquid from the wet area 25 in the direction of the edge part 15.

As in FIG. 4, the embodiment according to FIG. 6 can involve the double-folded first part 21 lacking holes and the double-folded second part 22 containing holes, with the associated advantages referred to in conjunction with the description of FIG. 4, that is to say increased air permeability at the same time as the first part 21 prevents loose material, for example SAP granules, from leaking out through the top layer 3. A further advantage is that the SAP granules that have migrated out through the holes 23 can continue to be active in the space 29 between the first and the second parts 21, 22.

Neither of the embodiments described above with double-folded first and second parts 21, 22 is subject to the constraint of having both the first and the second parts 21, 22 double-folded, but one of the parts can be unfolded or can be provided with multiple folds. As in FIGS. 3 and 5, the second part can also be shorter than the first part, or alternatively of identical length as described above.

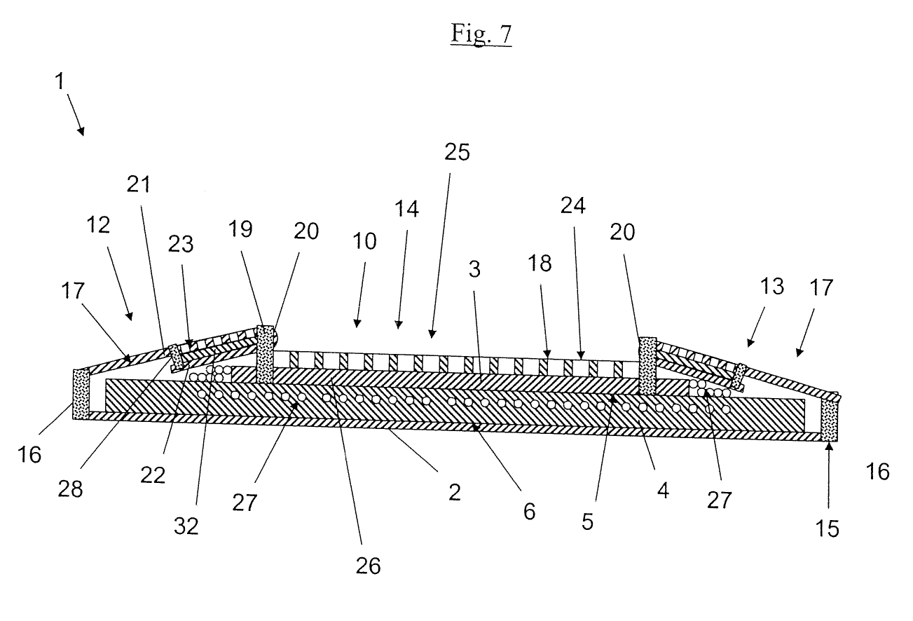

FIG. 7 depicts schematically a sectional view along the line A-A in FIG. 1 according to a sixth embodiment of the invention. FIG. 7 shows that the absorbent product 1 comprises a third material layer 32 positioned between the first part and the second part 21, 22 in the first material layer 17. The third material layer 32 can be an absorption body which absorbs liquid that has passed through the holes 23 in the first layer 17; or it can be an elastic element that can form the top layer, or an adhesive. FIG. 7 depicts the same embodiment as in FIG. 3, although the third material layer 32 can be positioned between the first and the second part in any of the embodiments shown in FIGS. 2-6.

The invention is not restricted to the embodiments indicated above, but can be varied within the scope of the accompanying Patent Claims. It can be mentioned here by way of example that the absorbent product can lack a receiving layer, in which case the top layer does not need to be attached to the receiving layer via the inner joining zones, but can be attached at the inner joining zones directly to the absorption body. Another example is that the absorbent product constitutes a panty liner intended to absorb only small quantities of liquid, for example discharges, etc., and for this reason consists only of a top layer and a water-impermeable backing layer. The top layer in this case is attached only to the backing layer in the inner joining zones and/or in the outer joining zones.

A further example is that the top layer is attached to the edge part of the backing layer only via the outer joining zones, and not to the rest of the absorbent product. The inner joining zones thus comprise only the first material layer and the second material layer.

A further example is that the first part can be shorter than the second part, in which case the first part can contain holes for a distance between the fold line on the second material layer and the interjacent joining zone.

1. An absorbent product comprising a top layer consisting of a first material layer and a second material layer, the first material layer being attached directly to the second material layer, the absorbent product exhibiting two lines extending in the longitudinal direction, which lines divide the product in the lateral direction in such a way that the product exhibits a first side zone, a second side zone and positioned between them a central zone,
wherein the first material layer comprises two subsidiary layers arranged to either side of the second material layer, each subsidiary layer comprising a first part and a second part, where
wherein the first part and/or the second part comprises openings,
wherein the first material layer is folded over itself at least once such that the first part is folded over the second part along a fold line,
wherein the first part has an extension in a lateral direction being less than or equal to or larger than the extension of the second part in the lateral direction,
wherein at least one of the parts is arranged to prevent loose particles present under the top layer from migrating out from the absorbent product via the openings, the first material layer being attached directly to the second material layer adjacent to the lines extending in the longitudinal direction via an inner joining zone being adjacent the fold lines.

2. The absorbent product according to claim 1, wherein the first material layer comprises two subsidiary layers arranged to either side of the second material layer, each subsidiary layer comprising a first part and a second part, where the first part and/or the second part contains openings, and where the first part is folded over the second part along a fold line, the first material layer being attached directly to the second material layer adjacent to the lines extending in the longitudinal direction in inner joining zones.

3. The absorbent product according to claim 1, wherein the first part of the first material layer contains openings and in that the second part of the first material layer lacks openings, which first part is folded over the second part in such a way that the openings are covered by the second part, the first part being arranged on the absorbent product in such a way that it faces towards a wearer during use.

4. The absorbent product according to claim 1, wherein the second part contains the openings and where the first part lacks openings, which first part is folded over the second part in such a way that the openings are covered by the first part, the first part being arranged on the absorbent product in such a way that it faces towards a wearer during use.

5. The absorbent product according to claim 1, wherein both the first part and the second part contain the openings, which first part is folded over the second part in such a way that the openings in the first part partially overlap the openings in the second part, the first part being arranged on the absorbent product in such a way that it faces towards a wearer during use.

6. The absorbent product according to claim 1, wherein the first part has an extent in the lateral direction that is smaller than the second part, or that the second part has an extent in the lateral direction that is smaller than the first part.

7. The absorbent product according to claim 1, wherein the absorbent product comprises a receiving layer, to which the first material layer and the second material layer are attached in the inner joining zone.

8. The absorbent product according to claim 1, wherein the absorbent product comprises a backing layer, to which the first material layer is attached by means of an outer joining zone adjacent to a surrounding edge part which surrounds the absorbent product.

9. The absorbent product according to claim 8, wherein the absorbent product comprises an absorption body positioned between the backing layer and the top layer.

10. The absorbent product according to claim 1, wherein the openings comprise holes and/or slits.

11. A method for the manufacture of an absorbent product, the absorbent product comprising a top layer containing a first material layer and a second material layer, which first material layer is attached directly to the second material layer, the absorbent product exhibiting two lines extending in the longitudinal direction, which divide the product in the lateral direction in such a way that the product exhibits a first side zone, a second side zone and positioned between them a central zone,
wherein the first material layer comprises two subsidiary layers arranged to either side of the second material layer, each subsidiary layer comprising a first part and a second part,
wherein the first part and/or the second part comprises openings,
the method comprising:
folding the first material layer over itself at least once such that the first part is folded over the second part along a fold line,
wherein the first part has an extension in a lateral direction being less than or equal to or larger than the extension of the second part in the lateral direction, wherein at least one of the parts is arranged to prevent loose particles present under the top layer from migrating out from the absorbent product via the openings,
wherein the first material layer is attached directly to the second material layer adjacent to the lines extending in the longitudinal direction via an inner joining zone being adjacent the fold lines.

12. The method according to claim 11, wherein the first material layer comprises two subsidiary layers arranged to either side of the second material layer, each subsidiary layer comprising a first part and a second part, where the first part and/or the second part contains the openings, and where the first part is folded over the second part along a fold line, after which the first material layer is attached directly to the second material layer adjacent to the lines extending in the longitudinal direction by means of inner joining zones.

13. The method according to claim 11, wherein the first part of the first material layer comprises the openings, and where the second part of the first material layer lacks openings, and where the first part is folded over the second part in such a way that the openings are covered by the second part, the first part being arranged on the absorbent product in such a way that it faces towards a wearer during use.

14. The method according to claim 11, wherein the second part comprises the openings, and where the first part lacks openings, and where the first part is folded over the second part in such a way that the openings are covered by the first part, the first part being arranged on the absorbent product in such a way that it faces towards a wearer during use.

15. The method according to claim 11, wherein both the first part and the second part comprise the openings, where the first part is folded over the second part in such a way that the openings in the first part partially overlap the holes in the second part, the first part being arranged on the absorbent product in such a way that it faces towards a wearer during use.

16. The method according to claim 11, wherein the first part is made shorter in the lateral direction than the second part, or that the second part is made shorter in the lateral direction than the first part.

17. The method according to claim 11, wherein the absorbent product comprises a receiving layer, to which the first material layer and the second material layer are attached via the inner joining zone.

18. The method according to claim 11, wherein the absorbent product comprises a backing layer, to which the first material layer is attached via an outer joining zone.

19. The method according to claim 18, wherein an absorption body is positioned between the backing layer and the top layer.

20. The method according to claim 11, wherein openings contain holes and/or slits.
